(19) United States
(12) Patent Application Publication
(10) Pub. No.: US 2025/0160980 A1
(43) Pub. Date: May 22, 2025

(54) SYNCHRONIZATION OF THE OPERATIONAL ENVELOPES OF INDEPENDENT SURGICAL DEVICES

(71) Applicant: Cilag GmbH International, Zug (CH)

(72) Inventors: Frederick E. Shelton, IV, Hillsboro, OH (US); Jacqueline Corrigan Aronhalt, Loveland, OH (US); Kevin M. Fiebig, Cincinnati, OH (US); Taylor W. Aronhalt, Loveland, OH (US)

(21) Appl. No.: 18/810,133

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. A61B 34/37; A61B 34/00; A61B 34/20

(52) U.S. Cl. CPC A61B 34/37; A61B 34/20; A61B 34/25; A61B 34/74; A61B 2034/742

(57) ABSTRACT

Devices, systems, and techniques for synchronization of the operational envelopes of independent surgical devices. An example device may receive a user surgical control input. The device may send an indication of a preferred operational envelope to a negotiating surgical device. The device may constrain operation responsive to the user surgical control input based on a reduced operational envelope that is determined by a response, from the negotiating surgical device, to the indication of the preferred operational envelope. The reduced operational envelope may result in a greater constraint on the operation responsive to the user surgical control input than the preferred operational envelope.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

This application is related to the following, filed contemporaneously, the contents of each of which are incorporated by reference herein:
U.S. patent application Ser. No. 18/809,890, filed Aug. 20, 2024, and
U.S. patent application Ser. No. 18/810,170, filed Aug. 20, 2024.

### BACKGROUND

With the complexity and autonomy of smart devices, particularly in the medical field, interactions may be managed between multiple smart devices (e.g., and legacy devices). Systems may operate in isolation or with limited collaboration, limiting their effectiveness and potentially leading to instability or predictability failures. Means for coordinating these systems may be static and may not adapt based on changing circumstances or patient parameters, posing a potential challenge in providing patient care and monitoring.

### SUMMARY

In operating rooms, multiple surgical devices may operate in close proximity to one another. In addition, the devices may all be from different manufacturers and may have different control systems. The devices may not be aware of the presence of other devices. Even if the devices are aware of other devices, the devices may not be able to communicate to coordinate their actions. This lack of coordination may cause the surgical devices to become entangled with each other and, in the worst-case scenario, injure a patient.

Feature(s) described herein relate to the synchronization of surgical device operational envelopes. In this case, although the precise movements of different devices are not synchronized, the devices may maintain synchronized operational areas, so as to avoid unwanted interaction between the devices. The shape or location of an operational envelope may be altered. For example, the operational envelope of a first device may change based on a user actively modifying the operational envelope of second device and/or based on the second device's movement. A (pre)defined balance between the two operational envelopes may be maintained by altering one when the other is modified (e.g., by the active control of the user). The operational envelopes may be synchronized by changing the loci of actions or functional limits of a first system based on the movements of a second (e.g., autonomous) system.

In an example, two robotic arms may be operating in the same area of a patient. To avoid the robotic arms becoming tangled, the area in which each arm is able to move may be bounded. In another example, the robotic arms may be configured to maintain at least a minimum distance from one another. In yet another example, the robotic arms may communicate with one another to negotiate for space (e.g., if one arm needs to move into the operational envelope of the other to perform a step in a surgical procedure).

### BRIEF DESCRIPTION OF THE DRAWINGS

Examples described herein may include a Brief Description of the Drawings.

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 illustrates an example layout of a surgical operating room.

FIG. 6 is a block diagram illustrating example processing modes in a surgical device.

FIG. 7 illustrates an example of overlapping zones of movement between devices in a surgical operating room.

FIG. 8 is a block diagram illustrating an example of optimizing device movement based on a constraint.

FIG. 9A illustrates an example API between surgical instruments.

FIGS. 9B and 9C illustrate an example of a scope maintaining a target device in a field of view as the target device moves.

FIG. 10 illustrates an example method that may be performed by a surgical instrument.

### DETAILED DESCRIPTION

A more detailed understanding may be had from the following description, given by way of example in conjunction with the accompanying drawings.

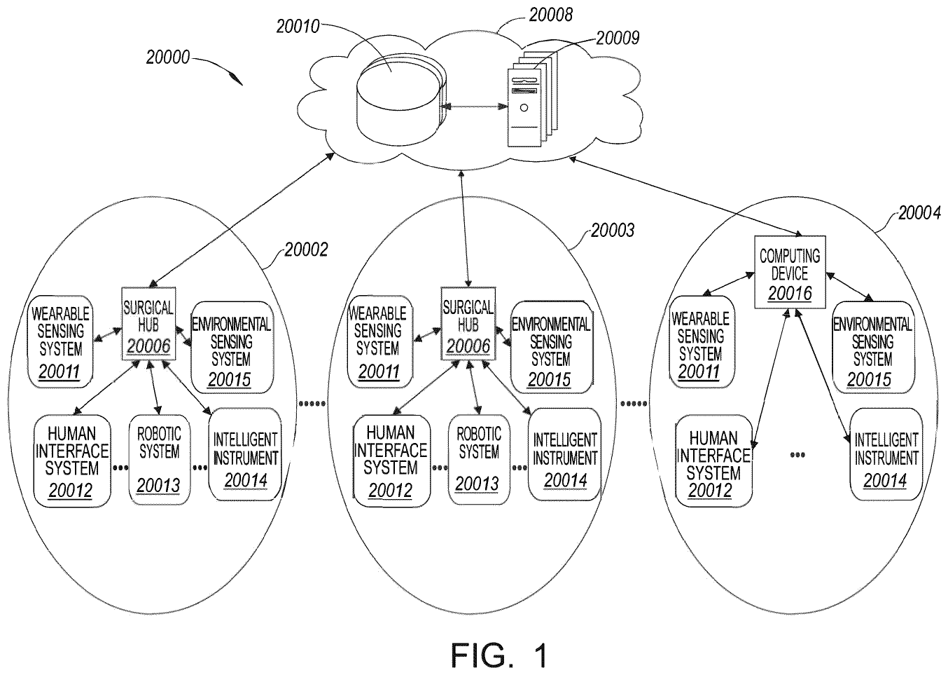

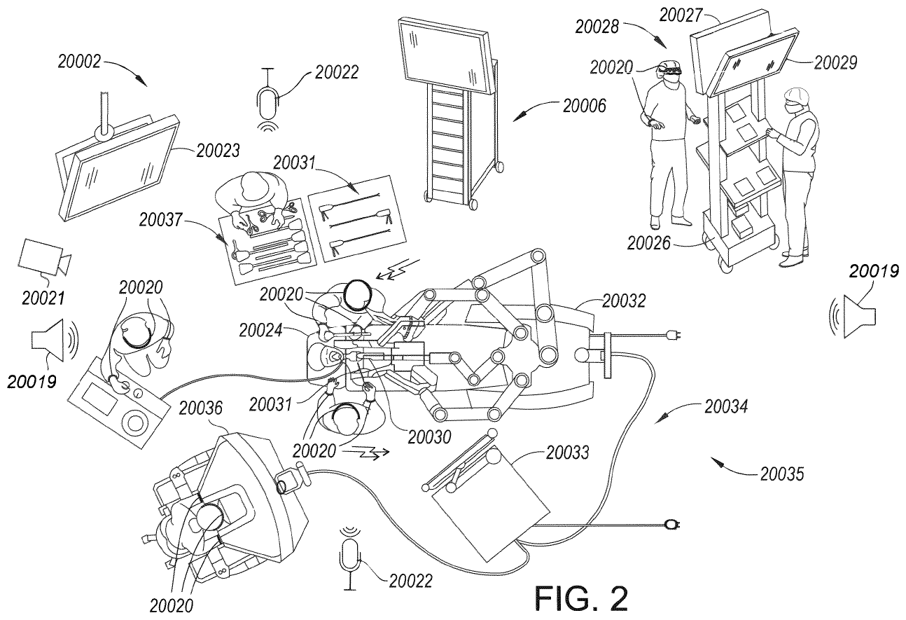

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

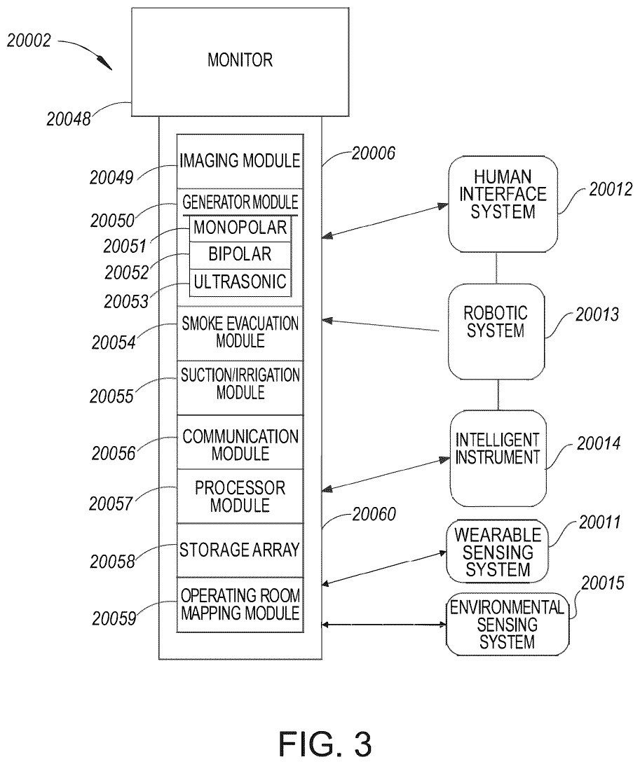

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

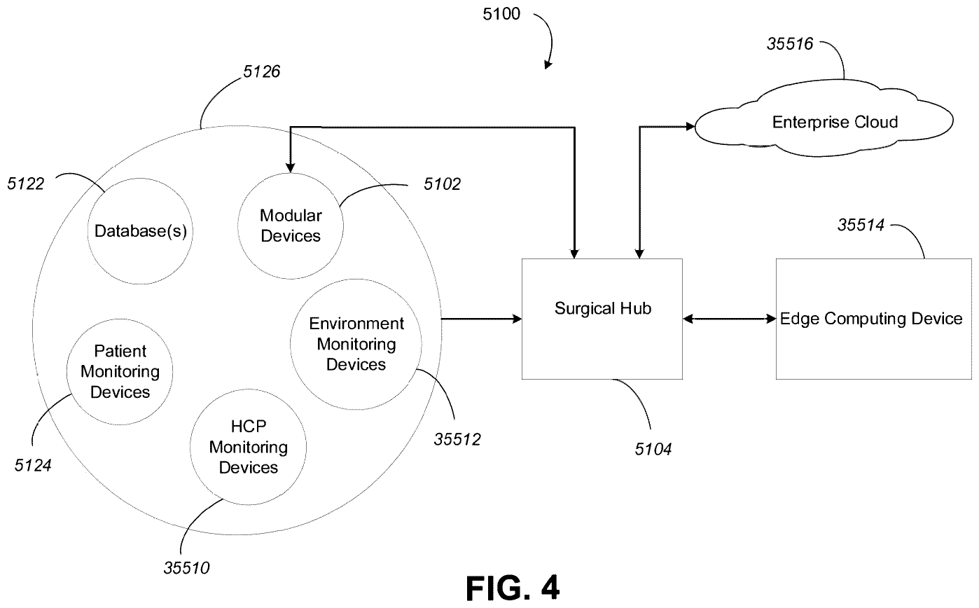

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

In operating rooms, multiple surgical devices may operate in close proximity to one another. In addition, the devices may all be from different manufacturers and may have different control systems. The devices may not be aware of the presence of other devices. Even if the devices are aware of other devices, the devices may not be able to communicate to coordinate their actions. This lack of coordination may cause the surgical devices to become entangled with each other and, in the worst case scenario, injure a patient.

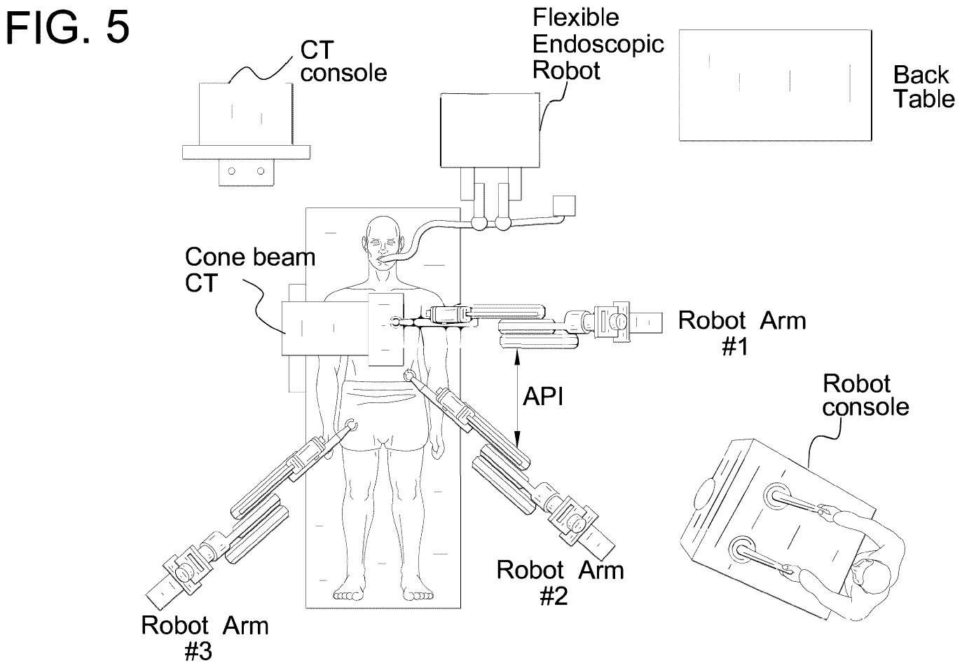

FIG. 5 illustrates an example surgical operating room in which several different devices are present. Some of the devices may be “smart” surgical devices (e.g., robotic arms, smart endoscope, etc.) while other devices may be legacy devices (e.g., a CT machine) that do not have the same level of awareness as the smart devices. The legacy devices may not be able to communicate with any other device, whereas the smart devices may have communication capabilities.

The operational areas of activities by a first system and a second system may be synchronized. The systems may have synchronized devices that are separately controlled by the systems. The individual actions of a first device to a second device may not be synchronized. For example, the shape or location of a operational smart device envelope may be altered (e.g., passively altered). For example, the operation envelope may be altered based on the active modification (e.g., by a user) of another related smart device movement. A predefined balance between two operations envelopes may be used to determine how the operational envelope(s) are altered. For example, an operational envelope may be altered when the other operational envelope is modified by the active control of the user. For example, the loci of actions or envelope of functional limits of a first system may be changed based on the movements of a second autonomous system.

The operational envelope of the motions of a first system may be adapted based on the motions, activities, and/or location of another device (e.g., a moving smart device). For example, the activities, movements, and/or operation of a first smart system may cause a second system to change its operational envelope (e.g., through the second system's own decisions and operations). The second system may not be told whether to operate in a predefined space.

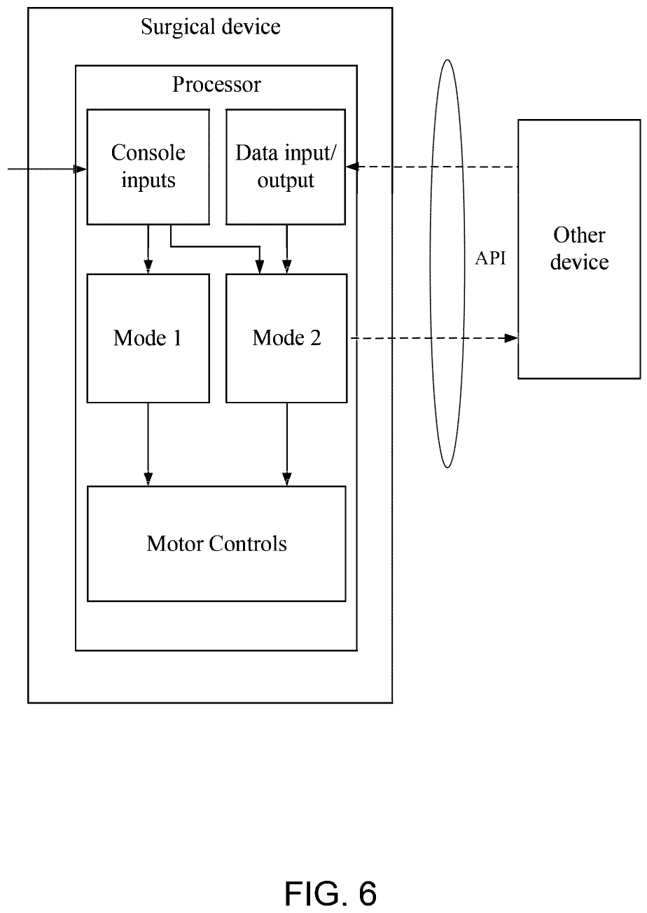

FIG. 6 is a block diagram illustrating example processing modes in a surgical device (e.g., a robotic arm). The surgical device may have a processor. The processor may receive control inputs (e.g., user control input from a console controlled by a user) and/or data input from another device (e.g., via an API). For example, the user surgical control input may include at least one of: moving a joystick, squeezing a trigger, touching a display, pressing a button, flipping a switch, turning a dial, and/or the like.

If the surgical device is in a first mode (e.g., Mode 1), the device may only use the console inputs to determine the device's movements (e.g., motor controls). That is, the device may be completely controlled by the user. For example, the surgical device may perform an operation responsive to the user surgical control input. The operation may include at least one of: actuation of joints external to a patient's body, a position of the surgical device, displaying an image on a display, and/or the like.

If the surgical device is in a second mode (e.g., Mode 2), the device may consider the console inputs and the data input from the other device when determining the device's movements. For example, the user may direct the surgical device to move a robotic arm to a new area of the patient. The surgical device may know (e.g., from the data input from the other device) that the other device is in that area. In this case, the surgical device may indicate a warning to the user (e.g., indicating a potential collision between the devices). The surgical instrument may know that it would have to cross over where the other device is located to reach that area. In this case, the surgical device may constrain its operation to avoid collision between the surgical device and the other (e.g., negotiating) surgical device. For example, the surgical device may determine a different path to take to the area than the path originally indicated by the user. The different path may be calculated to avoid a collision between the two devices, while still allowing the surgical device to reach the indicated area of the patient.

The data provided between multiple (e.g., two) moving systems may be used to determine the operational space of the systems. One or more of the system(s) may use the data to adjust its functional space, orientation, position, etc. (e.g., based on the monitored changes in the other system). The adjustments may be, for example, follow-along, preemptive repositioning to avoid anticipated confrontational use of occupied space, avoidance of an electrical interference zone, and/or changes in orientation to improve the functional interactions between the systems.

In an example, multiple separate robot arms may be used to retract and dissect an attachment of an organ (e.g., the stomach or other organ with interrelationships to other organs or fixation to the substructure of the body). A first system (e.g., robot arm) may be used to retract a portion of the organ (e.g., stomach) in the laparoscopic space via a percutaneous insertion point. A second system (e.g., instrument) may be used to generate forces between the first system (e.g., through the tissue) to dissect the tissue. The second system may constantly provide the first system position, orientation, and/or force direction information. The first system may use this information to reposition its external arm position to provide the best stable counter force for the second system. The systems may not be at risk of colliding. The user may not have given a direct command for the first system to move. The second system may determine that the first system repositioned itself to provide better support to the second system.

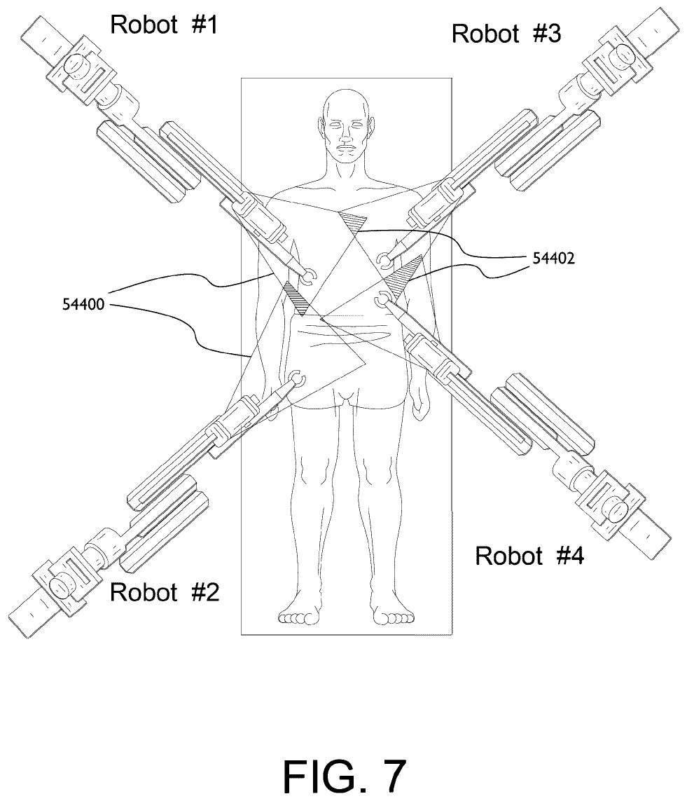

As illustrated in FIG. 7, multiple Hugo robot arms may be used simultaneously during an operating procedure. Each robot arm may have acceptable operation zone(s) (e.g., both inside and outside of the patient's body). Other device(s) (e.g., a Monarch smart endoscope) may be introduced to the operating room. The system may inform the robot arms of the operational envelopes 54400 that overlap between the robot arms and other device(s). The overlaps may create shared air space 54402 between the operational zones, which are shown as shaded regions in FIG. 7.

One or more of the devices may attempt to reduce any shared air space 54402. The shared air space 54402 may be treated as a negotiated zone between the devices. One of the devices may send an indication of a preferred operational envelope to a negotiating surgical device. For example, if a first device intends to enter the shared air space (e.g., in response to a user surgical control input), the first device may send a request to a second device that is sharing the shared air space. The request may be used to determine a negotiation protocol between the devices. The first device may send an indication of the preferred operational envelope according to the negotiation protocol. The first device may determine to send the indication of the preferred operational envelope based on at least one of: a class of the surgical device, or a class of the second (e.g., negotiating) surgical device. The indication may further indicate one or more suggested reduced operational envelopes (e.g., in addition to the preferred operational envelope). The second device may send a response that indicates a selection of the reduced operational envelope.

The second device may respond to grant or deny the first device access to the shared air space. The second device may send a response that indicates a selection of the reduced operational envelope (e.g., from the options presented by the first surgical device). The second device may send a response that indicates a rejection of the preferred operational envelope and an instruction to use a different reduced operational envelope.

The first device may its constrain operation responsive to the user surgical control input based on a reduced operational envelope that is determined by the response (e.g., from the negotiating surgical device) to the indication of the preferred operational envelope. The reduced operational envelope may result in a greater constraint on the operation responsive to the user surgical control input than the preferred operational envelope. The greater constraint on the operation (e.g., operation responsive to the user surgical control input compared to the preferred operational envelope) may involve a movement restriction that limits a physical space that the surgical device may enter, a display restriction that restricts portions of the surgical device's field of view that may be displayed, a time restriction that limits times during which the surgical device has access to a physical space, and/or the like.

The response from the negotiating surgical device may be based on at least one of: an operational envelope of the negotiating surgical device, a present state of the negotiating surgical device relative to the preferred operational envelope, a procedure being performed using the surgical device, or a step in the procedure being performed using the surgical device.

In an example, the second (e.g., negotiating) surgical device may be a robotic surgical device and the operation responsive to the user surgical control input may be a robotic joint actuation. The preferred operational envelope may be a physical space that accommodates potential robotic joint positions. In this case, the first surgical device may constrain operation responsive to the user surgical control input (e.g., based on a reduced operational envelope) by restricting access of the first surgical device to a portion of the physical space.

Adaptive robot-to-robot no fly zones may be based on an aspect of a first robot arm (e.g., location of the second robot cart, its robot arm position, movements, required operational envelope, etc.). The no-fly zones may place limitations on a second robot arm. The second robot arm may receive the limitations from the first robot arm or a separate robot.

In an example, a first laparoscopic multi-cart robot may be used to for dissection and resection of a mid-parenchyma tumor (e.g., that is on the junction of two segments). A surgeon may want to avoid removing two full segments. The surgeon may attempt to separate the tumor from the artery and vein. The surgeon may determine (e.g., during surgery) that the tumor has invaded the bronchus. The surgeon may determine penetration depth and the extent of invasion (e.g., using a flexible endoscopy scope controlled with a separate robot). The introduction of the second robot may not involve repositioning an existing first robot cart. A cart positioned towards the head of the patient may have a working envelope outside of the body that encompasses some of the space occupied by the flexible endoscopy robot and its operating envelope.

Once operational, the second robot may establish communication with the first robot. The second robot may communicate its location and size dimensions. The second robot may define the space it intends to use (e.g., at a minimum) to operate and inform the first robot of the reduced operational envelope available in which the first robot can operate to avoid entanglement. This regulation of the first robot by the second robot may involve defining the space reduction and active monitoring of the first robot arm. The restriction may involve defining a portion of the full operating envelope in which the first robot can no longer operate. The restriction may be an actively adjusted regulation of the space (e.g., that changes as the two robot arm coordinate their operation with the flexible endoscopy robot).

The space may be reduced (e.g., only reduced) as needed for the second robot and the endoscopy robot to move. In this case, the first robot may be allowed to occupy shared space (e.g., as long as it does not intend to always be in that space). If the first and second robots intend to occupy the same shared space, the robots may negotiate (e.g., based on priority, user input, or computational ordering) to choreograph the robots' motions. This may allow the robots to move around each other (e.g., through a series of pre-calculated alternating motions to allow them to move around each other) without adverse interactions.

Smart system(s) may be able to identify the location of other smart systems relative to each other. In the previous example, as the flexible endoscopy robot is brought into the OR and set up, the user may input the location and operational window for operation or a smart system may define the location and operational envelopes of the robots (e.g., relative to each other).

The surgical hub and a room-based camera may be used to identify the exact location, shape, and operational window to be used by a device (e.g., based on the setup of the devices in the OR). For example, multiple perspective cameras with overlapping image coverage may be used. Fewer cameras may be used if, for example, light detection and ranging (Lidar) is used to detect distances and/or structured light is used to define shapes and volumes. The robot towers/carts may integrate laser alignment and Lidar positioning to define the location of the arms, carts, and control systems.

For example, a Hugo robot may use a laser alignment and positioning system to determine where its arms are relative to the patient. If an ion or Monarch flexible endoscopy robot is positioned in the OR, the Hugo robot may use the alignment system (e.g., and information from the flexible endoscopy system) to identify the location of the endoscopy system within the room and around the patient.

Physical docking locations or mechanical linkages may be used to place the movable robot carts and towers in known (pre)defined locations (e.g., relative to any stationary larger robot systems). For example, an Ottava table-based robot may have a docking location with a physical aligning and locking system. The aligning system may enable a monarch mobile flexible endoscopic robot to knows the location of the tower, and be placed around the tower accordingly.

The working envelope of the arms, instruments, and end-effectors of each of the smart system may be determined. Overlapping spaces of the operational envelopes as shared space may be identified, as shown in FIG. 7. The utilization of the shared space by each of the systems may be organized. Subtractive operational envelope reduction may be used to reduce the space a smart system is allowed to use (e.g., based on the need or priority of the other system to use that shared envelope).

If tight cooperative use of the shared space is planned, the two systems may develop a plan for sequential choreographed motions. The systems may determine which system will determine the choreographed motion plan. The systems may choose between multiple movement options (e.g., who moves first, whether to coordination will be similar to a multi move chess match, etc.).

Individualized reactive isolated step operation may be used for the robots to move one step at a time. At each step, the robots may reassess the new situation (e.g., rather than fully planning out multiple moves to choreograph together).

As illustrated in FIG. 7, the operational envelopes of the robots overlap each other. Robot #1's envelope may be reduced by the overlap with robot #2 and robot #3. Similarly, robot #4's and robot #3's envelopes may be reduced. In this case, the reduced envelopes may be due to an adjacent robot having a higher priority use of the space (e.g., so the operational envelope of the lesser priority robot is reduced). This rule may be reversed or the “shared” space may be reallocated (e.g., dynamically on-the-fly). An overlap may be partially owned by a system, or the sharing may be choreographed over time (e.g., as one moves out of the space, the other may move through the space). These shared motions may be stepwise (e.g., with each robot making one move, and then the other, for example, to better utilize the shared spaces).

The systems may define and regulate shared operation envelopes (e.g., inside and/or outside of the body). For example, in a thoracic procedure, a plurality of (e.g., five) Hugo robots may be arrayed around the patient with at least one accessing the space over the patient head. The operational envelope overlaps may be managed by one or more of the Hugo robot(s). A monarch device may be introduced to the OR next to the at least one Hugo robot by the head of the patient. This may create new (e.g., two new) overlap zones. A first overlap zone may be the space the Monarch deems necessary for operation (e.g., extension and retraction space). A second overlap zone may be a portion of the shared space that the monarch device or the Hugo robot is allowed to use (e.g., but not at the same time). The two robots on the left of the patient may be repositioned to allow for the flexible endoscopy robot (e.g., monarch) to be positioned by the head and the flexible endoscopy scope to be introduced in the mouth.

There may be a no-fly zone around the flexible endoscopy robot because it cannot move out of the way for the adjacent robot station to share the space. Accordingly, the robots must avoid that space. In the shared space, two arms of the same controlled robot overlap and therefore could collide, but the robot controller may limit the zone to only one robot arm at a time.

The no-fly zone may change over time (e.g., in a choreographed manner). In this case, the robot arms may consider alternate configurations to get a first arm out of the space through which another arm intends to move. Adaptive robot-to-robot no-fly zones may be determined based on the movement of one or more smart devices. The no-fly zones may be inside or outside the patient's body.

The activation state of a device may affect the viable operation envelope of another. For example, a first smart device's position, motions, articulation, energy potential, or activation state may cause the system to adjust adjacent smart end-effectors in near proximity.

For example, conductive end-effectors zone of occlusion and interaction may be modified (e.g., depending on whether one of the end-effector's monopolar energy is active, or is active and above a certain threshold. This may prevent inadvertent energizing of a non-monopolar device (e.g., based on either inadvertent contact or close proximity in contact with the same tissue where the second device could become part of the return path). The zone modification may minimize inadvertent burns to the patient away from the surgical site (e.g., based on the continuity path of the second device's other tissue contacts).

An aspect of an advanced energy device may be monitored. The monitored aspect may be used to derive an envelope within which are handling specification (e.g., associated with the surrounding tissue or the other instruments). The handling specifications may be used to define active envelopes of restricted operation. These envelopes may be static representations based on the monitored parameter. The envelopes may be adapted or morphed (e.g., based on second device composition implications). The adaptations may be performed if (e.g., under (pre)defined conditions) there are exceptions to a rule governing the envelope. The system may determine that certain devices (e.g., special insulations in the devices or the trocar) or certain circumstances (e.g., the user acknowledged a risk and proceeded anyway, certain tissue conditions, or device orientations) may cause the systems to interact more closely. In this case, the system may actively adjust the envelopes (e.g., on the fly).

The operational window that is adjusted due to the proximity of a second device may limit the approach of the second device if the first device is active. The system may wait to activate the first device if the second device is within a range from the first device. For example, the activation (e.g., energizing) of a first device may be limited if the first device is in close proximity to a second device that has sensing means that would be effected by the energy being used near the second device. For example, a sensing means may be affected by monopolar RF, microwave, or irreversible electroporation.

A system may adjust the usable input situation of another device to bound its operation. A (e.g., independent) system may define another system's operational envelope.

One or more functions of related systems that affect the physiologic parameter of the patient may be adjusted (e.g., if the physiological parameter is out of pre-established bounds). Multiple systems may be synchronized by defining preventative or allowable response to undesirable events.

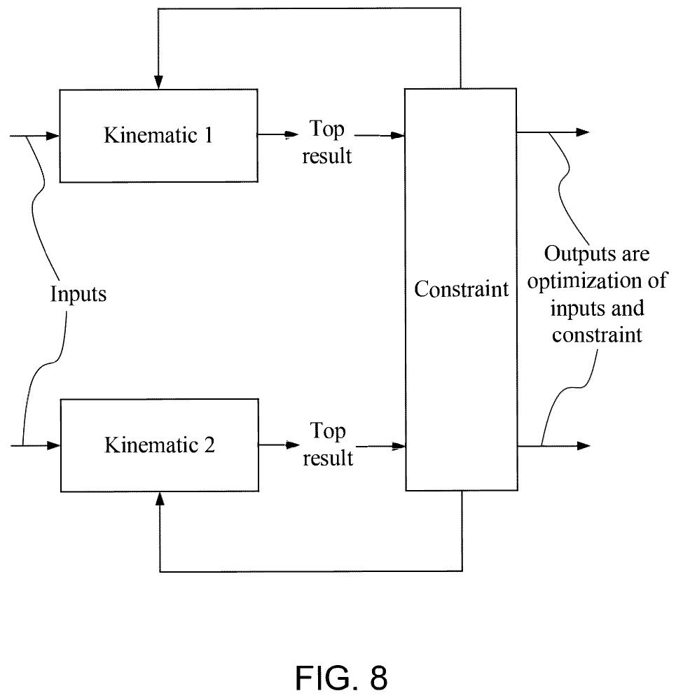

The operational envelope(s) may be adapted based on one or more constraints (e.g., real-time patient status/condition, surgeon usage, and/or adverse or undesirable events). For example, FIG. 8 illustrates an example block diagram illustrating a technique for optimizing outputs based on inputs and a constraint. As shown, one or more inputs may be considered by an operational envelope control device. The inputs may be sent through a first kinematic equation and a second kinematic equation. The kinematic equations may (e.g., each) output a top result.

The top results may be analyzed based on the constraint. For example, if the constraint is a no-fly zone for a robot arm and the top results would result in the robot arm entering the no-fly zone, the constraint may feedback this information to the kinematic equations. The kinematic equations may be re-calculated, based on the inputs and the information related to the constraint, to output new top results. The process may be repeated until one or more of the top results do not conflict with the constraint. This optimized result may be output to the robot arm to indicate how the robot arm should move. Accordingly, the device may constrain its operation by selecting a first kinematic solution (e.g., for joint positions of a robotic arm) that is different than a second kinematic solution associated with the preferred operational envelope.

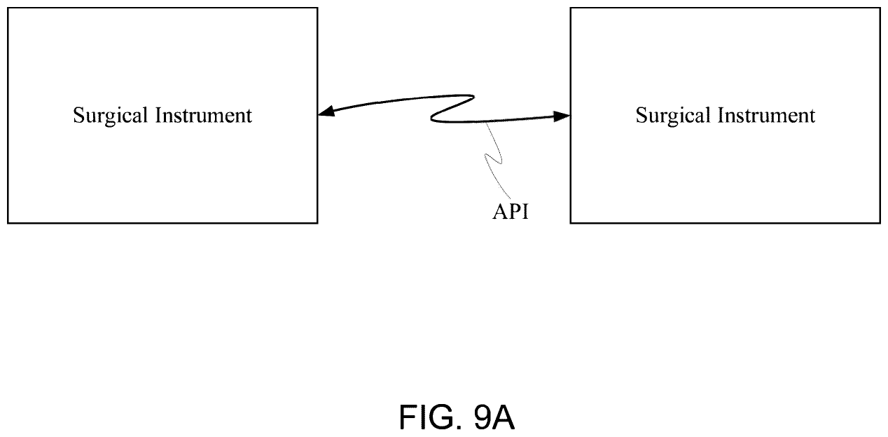

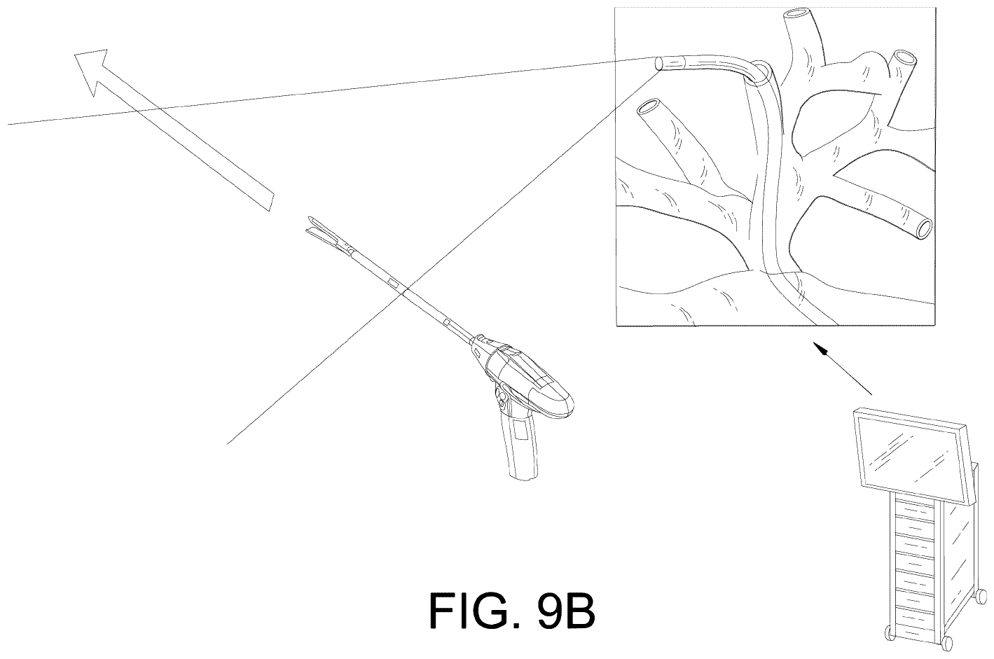

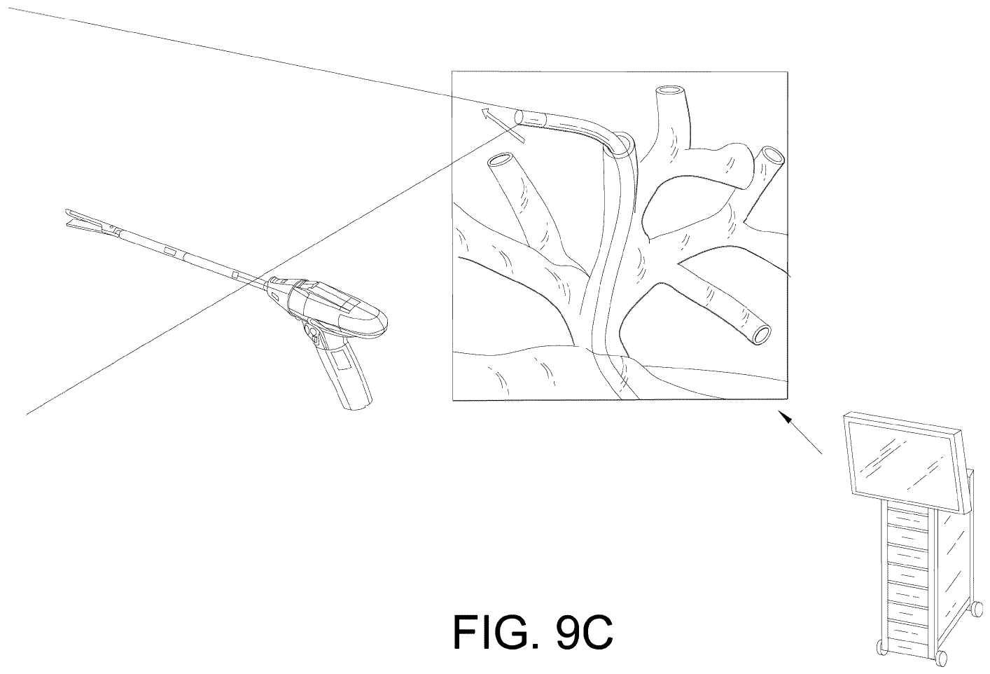

As illustrated in FIG. 9A, two or more surgical instruments may communicate with each other (e.g., via an API). In some examples, the operational envelope control device may be in one of the surgical devices, and that surgical device may output the determined results to the second surgical device to control the second device's movements.

The focus of a given imaging source may be adapted based on motions/actions of a first device and/or adverse reactions detected in the field of view. For example, as illustrated in FIGS. 9B and 9C, an imaging scope (e.g., an endoscope) may be configured to track the movement of a surgical instrument (e.g., a laparoscopic device). As shown, if the endoscope detects that the laparoscopic device has moved, the endoscope may automatically move to keep the laparoscopic device in the endoscope's field of view. The endoscope may be configured to track the laparoscopic device by another device (e.g., a surgical hub). The surgical hub may receive the configuration information from a user (e.g., a surgeon may indicate for the endoscope to track the laparoscopic device) and the surgical hub may forward that configuration information to the endoscope.

A smart visualization scope being able to track and move with the nexus of detected motion based on the instruments currently actively controlled by the user, could adjust the scope location, focal length, and field-of-view to follow the instrument end-effectors. The robotic laparoscopic hub may provide movement data to the scope to define the moving nexus of operation of the devices being controlled by the user. The scope may provide the data of its location and field of view.

The smart scope may (e.g., actively) act on the nexus location data it receives relative to the calculated center of field of view trying to keep the two measures synchronized. The robot console may use the field of view width and center to determine device locations and event outside of the field of view. The robot console may display those representations to the user to maintain peripheral awareness (e.g., while allowing the user to keep their field of view narrowed on the current action activities). Devices (e.g., each device) may control their own actions relative to the data it receives and the data it generates. The actions may not be synchronized with the motions of the other device in this case. The devices may provide data on their location and activities to the other so that the other may act accordingly to avoid issues (e.g., collisions or other interference).

Based on the movement of a surgical device (e.g., endocutter), the system may be able to track and predict where to position a camera so that the surgeon can maintain an uninterrupted field of view.

A scope with high resolution and/or digital zoom may be used to monitor the entire surgical field. The scope may only focus on a specific area at a given time. The scope may be positioned close to the body wall. The scope may have enough resolution to capture the entire internal abdominal cavity. An area of interest may be magnified (e.g., digitally), for example, instead of repositioning the scope, or optically adjusting the physical lens focus of the scope. The digital focus of the image, and what is visible to the user may be controlled manually by the user, or based on synchronized motions with other devices, as described herein.

By using digital and/or optical zoom and tracking, the entire surgical field may be monitored in the background while the user is able to focus on specific areas. With the entire surgical field being monitored by the system, alerts to the user and/or automatic refocusing of the image may help manage adverse reactions (e.g., bleeding).

For example, in a sleeve gastrectomy, delayed bleeding of the staple line may occur (e.g., several minutes after the stapling event when the surgeon is focused on other parts of the procedure). The imaging system may track and focus the camera based on the endocutter in use. As the surgeon moves between firings, the endocutter may be kept in focus so the surgeon can see the current transection. In an example, while the surgeon is working on the fourth firing of the sleeve, the first firing may start to bleed. The first firing may be outside the field of view of the current focus. Because the imaging system is tracking the entire surgical field, but focusing the visible image on the endocutter, the system may identify that bleeding is occurring. The system may send an alert to the user indicating that bleeding is occurring off screen. Reaction types may include a text-based alert warning of off-screen bleeding, a picture-in-picture showing the bleeding, an automatic refocusing of the image to zoom out and show the entire surgical field to include the bleeding, an alert based on a severity of bleeding or other adverse reaction (e.g., critical vs. nuisance bleeding may be picture-in-picture vs. text), and/or the like.

Visualization may be used as a bounding means for inhibiting actuations in obscured locations. Secondary data sources may be used to overcome obscured hazards to replace the obscured location.

The scope may be used to determine one or more portions of the visualization field inside of the patient that can be interacted with (e.g., manipulations, end-effector movement, energy device or endocutter repositioning, etc.) based on the ability to accurately visualize the spaces. Smoke from previous firings may occlude clear visualization of tissue. The scope may be used to constrain the actionable portions of the field based on what can be seen. This may be an absolute go/no go indication or a suggested no-fly zone with an override condition so that the user may enter the space if needed.

Data gathered from surrounding smart devices may be used to provide information (e.g., otherwise unavailable information) to another smart device. For example, that information may be otherwise unavailable due to lack of visibility in an obscured hazard.

For example, a pre-operative CT scan may indicate a series of metallic clips from a previous surgery. The pre-operative CT may utilize a surgical plan to indicate (e.g., in a real-time image) the projected location of the clips identified in the pre-op imaging. This may be done even if the body is in a different position during the surgery compared to during the full body CT. Data may provide fiducial location information associated with the locations of staples, bolts, plates, screws, and/or the like. If harmonic is used and a clip is accidentally clamped and energized, the blade may break. If a staple line is fired over a pre-existing clip and the blade of the staple contacts the clip, the blade may drag the staple along, damaging the staples its deploying. The exchange of data may be used to overcome the obscured hazard in these cases.

In another example, if a uterine manipulator is obscured in the laparoscopic view during a hysterectomy; the laparoscopic robotic controlled instruments may be limited from moving directly against the distal portion of the manipulator. For example, the laparoscopic instruments may (e.g., may only) move over or under the manipulator to create the separation planes to the bladder and rectum, respectively. After the dissection is complete (e.g., as manually entered by a user or based on pressure of uterine manipulator indicating free motion of uterus), the laparoscopic instruments may be unlocked from moving distally toward the cervix. The cooperation of laparoscopic energy and uterine manipulator colpotomy cup may be used to create a colpotomy.

The bounding means may be based on a configuration or state of the surgical site or organ (e.g., instead of the absences of direct visualization). For example, an endoluminal view (e.g., as in a colonoscope) may be checked by computer vision and compared to a bounding zone. The view within the bounding zone may indicate an ‘open’ lumen with proper balance of laparoscopic pressure and endoscopic pressure. If the lumen is occluded (e.g., smaller) than the bounding window, the endoscopic instrumentation may not deploy. The endoscopic instrumentation may be manually deployed (e.g., with Bluetooth) by a main system. The main system may be used for instrument identification and augmentation, which may be displayed on a user display (e.g., a main user interface control screen).

The operation envelope of a device may be adapted based on device performance, device error codes, alarms, and/or device capability. For example, if a user is using a harmonic device and the blade is running hot, the operational envelope may adjust to prevent undesired thermal spread. The operational envelope may be made smaller (e.g., immediately after the transection is made) until the blade cools to a temperature in which it won't damage the patient if inadvertent contact occurs. Once blade has cooled, the operational envelope of device may expand.

The operational envelope of a device may be adapted based on user performance. For example, if the user is using a harmonic device with continuous activation and a tiny tip, the shaft of the device may get warm. The operational envelope may adjust to protect critical structures from undesired thermal spread from the shaft.

In an example, a harmonic device may have a designated area of operation. Once staples have been deployed in a specific area, the designated area of operation for the energy device may be modified to exclude the area, the system may provide a warning to the user that metal is in that area (e.g., and that caution should be exercised), or an override condition may be necessary to activate the device in that area.

In an example, if unexpected, thin, diseased tissue is found on the intestines, the operational window of the areas available for graspers to grab may be updated to exclude this area.

Vision systems may be synchronized based on a specific tissue response and/or anatomy. If an endoscopic vision system and a laparoscopic vision system is used, a user may switch between cameras to view the moving system. The alternation of which camera is stationary or moving may be determined by a tissue response or selected anatomy.

In another example, after a low anterior resection (LAR), surgeons may use a colonoscope and a laparoscope to inspect the anastomosis for leaks. The colonoscope may be used to insufflate the colon and visualize the staple line intraluminally. The laparoscope may be used to inspect for air bubbles extraluminally from the abdominal cavity. If air bubbles are detected extraluminally, the colonoscope may orient the view to approximate the location of the air bubbles seen on the extraluminal side. Conversely, if bleeding is seen intraluminally, the laparoscope may orient its view to visualize the same extraluminal location.

At the end of an LAR, the surgeon may closely example the staple line internally to see the anastomosis through a colonoscope. At the same time, the laparoscope may be used to view the anastomosis from the abdominal cavity. The tracking may be used to look for bubbles due to fluid and/or air that entered into the system. In this case, it may be useful for the surgeon to be able to alternate between the laparoscope (e.g., identifying where the bubbles are coming out) and the colonoscope with a matching view from inside the colon.

Digital aspects (e.g., visualization, data exchange, processing, and/or monitoring) of devices may be synchronized. Devices may cooperatively exchange and process digital data to provide improved (e.g., optimal) operational envelope data.

Interference avoidance between visualization systems may be improved (e.g., by selectively adjusting imaging control for multiple systems to combine their outputs into a useable overlay). Discrete frequency spectral imaging may be used. For example, discrete frequency spectral imaging may be used to prevent inadvertent leakage of one frequency imaging into the other's domain. Structured light may use infrared laser spectrum to project a matrix array onto organs (e.g., to determine volumes and shapes. Because the structed light operates in this known spectral range, a multi-spectral camera may filter or adjust its normal imaging of infrared to filter out or avoid the added energy provided by the structured light. The multi-spectral camera may avoid the use of certain wavelengths, place the system temporally out of phase with the structured light projector, and/or filter out the additive aspect from the projector.

Multi-frequency sweeping scanning may be used to avoid interference between visualization systems. A first scan's sweeps may affect another scan's measurements or induce a local physiologic response, field sweeping scanning arrays, laser Doppler flowmetry (LDF), blood flow, etc. To avoid this interference, tissue impedance, ultrasound reflection, and/or sweeping scanning arrays may be staged out of phase with each other.

Multi-intensity imaging may be used to avoid interference between visualization systems. For example, CT resolution and/or reflection may be used. The multi-intensity imaging may be used depending on the material of an object of interest (e.g., metal, plastic, ceramic, bone, soft tissue, calcification, etc.). Reflective monochromatic light may be used to avoid interference between visualization systems. Surface conditions and/or shadows (e.g., reflectivity) may be used to avoid interference between visualization systems.

The intensity of a system may be adjusted to filter out interactions between systems. The intensity of a system may be adjusted to better visualize portions of the patient that another system is having trouble viewing (e.g., due to the energy used and/or the location to be imaged).

Aspects of separate visualization systems may be linked or coupled to provide complimentary data. For example, the Neuwave ablation confirmation system “Set-up CT scans” may use previous scans and/or scans completed at the start of a procedure. During a “Target” phase, the system may use the scans to define the target ablation and select the desired margins and/or zone to set on the tumor (e.g., based on tumor location to critical structures that may alter the margin/zone). The user may place one or more probe(s) in location(s) determined based on the CT scans. The location(s) of the probe(s) may be selected based on a best path for access. Ultrasound may be used for guidance (e.g., into a tumor). Ultrasound may be used to control the depth based off information from the CT scans. After the probes are placed, another CT scan may be used to confirm the probe(s) are at the target location(s). This process may be repeated until the probe(s) are at the planned location(s) (e.g., within the tumor).

Patient movement, breathing, etc. may alter the targeted probe location and actual probe location. The ultrasound guidance may increase the likelihood of proper placement (e.g., with minimal probe replacements). The system may register the set-up scans and probe scans to overlay. The registration may indicate the initial tumor scan and probe placement scan to the user. The registration may allow the user to change the ablation zone prior to ablating. The user may ablate and perform a final CT scan. The final CT scan may be overlayed with the initial scan to compare the actual ablation to the intended ablation. The ablation may be continued if the initial ablation did not fully ablate the intended ablation zone.

If a full thickness resection is completed with assistance from laparoscopic and endoscopic devices, the light from one side may wash out the view from the other side. A common system may monitor and control the illumination and vision systems to avoid this problem. If a non-visible signal (e.g., such as NIR wavelength >800 nm) is detected on a first side, the system may turn off the light on a second side to improve the view from the first side.

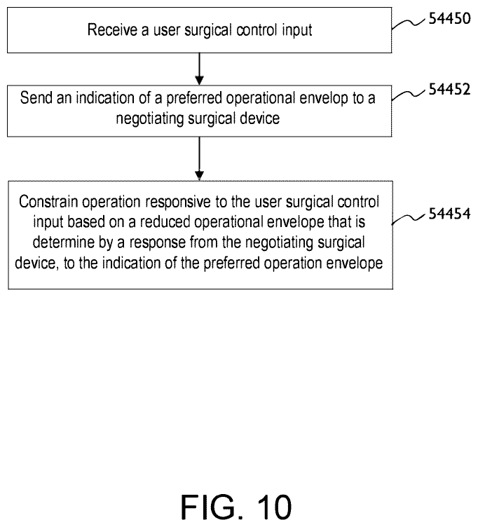

FIG. 10 illustrates an example method that may be performed by a surgical instrument. As shown, the method may involve, at 54450, receiving a user surgical control input. The method may involve, at 54452, sending an indication of a preferred operational envelope to a negotiating surgical device. The method may involve, at 54454, constraining operation responsive to the user surgical control input based on a reduced operational envelope that is determined by a response, from the negotiating surgical device, to the indication of the preferred operational envelope. The reduced operational envelope may result in a greater constraint on the operation responsive to the user surgical control input than the preferred operational envelope

What is claimed:

1. A surgical device comprising:
a processor configured to:
receive a user surgical control input;
send an indication of a preferred operational envelope to a negotiating surgical device; and
constrain operation responsive to the user surgical control input based on a reduced operational envelope that is determined by a response, from the negotiating surgical device, to the indication of the preferred operational envelope, wherein the reduced operational envelope results in a greater constraint on the operation responsive to the user surgical control input than the preferred operational envelope.

2. The surgical device of claim 1, wherein the processor being configured to constrain operation comprises the processor being configured to select a first kinematic solution for joint positions that is different than a second kinematic solution associated with the preferred operational envelope.

3. The surgical device of claim 1, wherein constraining operation avoids collision between the surgical device and the negotiating surgical device.

4. The surgical device of claim 1, wherein the processor is further configured to:
send a request to determine a negotiation protocol; and
send the indication of the preferred operational envelope according to the negotiation protocol.

5. The surgical device of claim 1, wherein the processor is further configured to determine to send the indication of the preferred operational envelope based on at least one of: a class of the surgical device, or a class of the negotiating surgical device.

6. The surgical device of claim 1, wherein: the negotiating surgical device is a robotic surgical device, the operation responsive to the user surgical control input is a robotic joint actuation, the preferred operational envelope is a physical space that accommodates potential robotic joint positions, and the processor being configured to constrain operation responsive to the user surgical control input based on a reduced operational envelope comprises the processor being configured to restrict access of the surgical device to a portion of the physical space.

7. The surgical device of claim 1, wherein the response, from the negotiating surgical device, is based on at least one of:
an operational envelope of the negotiating surgical device,
a present state of the negotiating surgical device relative to the preferred operational envelope,
a procedure being performed using the surgical device, or
a step in the procedure being performed using the surgical device.

8. The surgical device of claim 1, wherein the indication further indicates one or more suggested reduced operational envelope, and wherein the response indicates a selection of the reduced operational envelope.

9. The surgical device of claim 1, wherein the response, from the negotiating surgical device, indicates a rejection of the preferred operational envelope and an instruction to use the reduced operational envelope.

10. The surgical device of claim 1, wherein:
the user surgical control input comprises at least one of: moving a joystick, squeezing a trigger, touching a display, pressing a button, flipping a switch, or turning a dial;
the operation responsive to the user surgical control input comprises at least one of: actuation of joints external to a patient's body, a position of the surgical device, displaying an image on a display; or
the greater constraint on the operation responsive to the user surgical control input than the preferred operational envelope comprises at least one of: a movement restriction that limits a physical space that the surgical device may enter, a display restriction that restricts portions of the surgical device's field of view that may be displayed, or a time restriction that limits times during which the surgical device has access to a physical space.

11. A method performed by a surgical device, the method comprising:
receiving a user surgical control input;
sending an indication of a preferred operational envelope to a negotiating surgical device; and
constraining operation responsive to the user surgical control input based on a reduced operational envelope that is determined by a response, from the negotiating surgical device, to the indication of the preferred operational envelope, wherein the reduced operational envelope results in a greater constraint on the operation responsive to the user surgical control input than the preferred operational envelope.

12. The method of claim 11, wherein constraining operation comprises selecting a first kinematic solution for joint positions that is different than a second kinematic solution associated with the preferred operational envelope.

13. The method of claim 11, wherein constraining operation avoids collision between the surgical device and the negotiating surgical device.

14. The method of claim 11, wherein the method further comprises:
sending a request to determine a negotiation protocol; and
sending the indication of the preferred operational envelope according to the negotiation protocol.

15. The method of claim 11, wherein the method further comprises determining to send the indication of the preferred operational envelope based on at least one of: a class of the surgical device, or a class of the negotiating surgical device.

16. The method of claim 11, wherein:
the negotiating surgical device is a robotic surgical device,
the operation responsive to the user surgical control input is a robotic joint actuation,
the preferred operational envelope is a physical space that accommodates potential robotic joint positions, and
constraining operation responsive to the user surgical control input based on a reduced operational envelope comprises restricting access of the surgical device to a portion of the physical space.

17. The method of claim 11, wherein the response, from the negotiating surgical device, is based on at least one of:
an operational envelope of the negotiating surgical device,
a present state of the negotiating surgical device relative to the preferred operational envelope,
a procedure being performed using the surgical device, or
a step in the procedure being performed using the surgical device.

18. The method of claim 11, wherein the indication further indicates one or more suggested reduced operational envelope, and wherein the response indicates a selection of the reduced operational envelope.

19. The method of claim 11, wherein the response, from the negotiating surgical device, indicates a rejection of the preferred operational envelope and an instruction to use the reduced operational envelope.

20. The method of claim 11, wherein:
the user surgical control input comprises at least one of: moving a joystick, squeezing a trigger, touching a display, pressing a button, flipping a switch, or turning a dial;
the operation responsive to the user surgical control input comprises at least one of: actuation of joints external to a patient's body, a position of the surgical device, displaying an image on a display; or
the greater constraint on the operation responsive to the user surgical control input than the preferred operational envelope comprises at least one of: a movement restriction that limits a physical space that the surgical device may enter, a display restriction that restricts portions of the surgical device's field of view that may be displayed, or a time restriction that limits times during which the surgical device has access to a physical space.
